(19) United States
(12) Patent Application Publication
(10) Pub. No.: US 2009/0240064 A1
(43) Pub. Date: Sep. 24, 2009

(54) CRYSTALLINE FORM OF ATORVASTATIN HEMI-CALCIUM

(76) Inventors: Venkata Panakala Rao Gogulapati, Secunderabad (IN); Ramdas Chavakula, Secunderabad (IN); Mohan Bandari, Secunderabad (IN); Seeta Ramanjaneyulu Gorantla, Secunderabad (IN)

Correspondence Address: SCHWEITZER CORNMAN GROSS & BONDELL LLP, 292 MADISON AVENUE - 19th FLOOR, NEW YORK, NY, 10017, US

(21) Appl. No.: 12/280,263

(22) PCT Filed: Feb. 20, 2007
(86) PCT No.: PCT/IN2007/000062

§ 371 Date: Aug. 21, 2008

(30) Foreign Application Priority Data
Feb. 22, 2006 (IN) 291/CHE/2006

(51) Int. Cl. C07D 207/30

(52) U.S. Cl. 548/537

(57) ABSTRACT

The present invention relates to a new crystalline form of Atorvastatin hemi-calcium and also relates to a process for preparation of the same by treating Atorvastatin hemi-calcium amorphous form or form-I or mixture of amorphous and crystalline forms with methanol.

### FIELD OF THE INVENTION

The present invention relates to a novel crystalline polymorphic form of Atorvastatin hemi-calcium and the process for preparation of the same.

### BACKGROUND OF THE INVENTION

Atorvastatin hemi-calcium is known by the chemical name {[R-(R*,R*)]-2-(4-Fluorophenyl)-β,δ-dihydroxy-5-(1-methylethyl)-3-phenyl-4-[(phenylamino)carbonyl]-1H-pyrrole-1-heptanoicacid} calcium salt (2:1). Atorvastatin has the following formula.

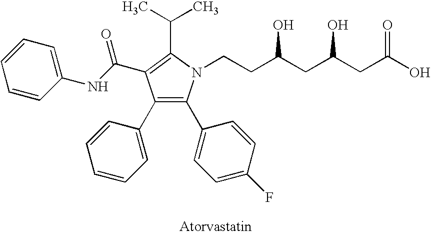

Atorvastatin hemi-calcium trihydrate, (βR, δR)-2-(4-Fluorophenyl)-β, δ-dihydroxy-5-(1-methyl ethyl)-3-phenyl-4-[(phenylamino)carbonyl]-1H-pyrrole-1-heptanoic acid-calcium salt trihydrate has been marketed as Lipitor, used for the inhibition of biosynthesis of cholesterol.

U.S. Pat. No. 4,681,893 first disclosed and claimed Atorvastatin. U.S. Pat. No. 5,273,995 disclosed Atorvastatin hemi calcium salt and it also disclosed the process for preparation of Atorvastatin hemi calcium salt by hydrolysis of lactone with sodium hydroxide in aq.methanol and saltification with aqueous calcium chloride.

Several crystalline forms of Atorvastatin calcium such as form I, II, III, IV, V, VI to XIX are disclosed in U.S. Pat. No. 5,969,156, U.S. Pat. No. 6,121,461 and U.S. Pat. No. 6,605,729 assigned to Warner-Lambert Co. U.S. Pat. No. 6,605,636 and U.S. Pat. Application 2002/0183378 assigned to Teva discloses the cryatlline forms VI, VII, VIII, IX, X, XI and XII. U.S. Pat. No. 6,867,306 assigned to Biocon discloses crystalline form-V. U.S. Pat. Application 2003/0114686 filed by Teva discloses forms X, A, B, B2, C, D & E. Crystalline forms VI & VII are disclosed in U.S. Pat. Application 2004/242899 filed by Dr Reddy Labs. Crystalline form F is disclosed in U.S. Pat. Application 2004/106670 filed by Teva. PCT publications WO 2005/090301, 2003/022053, 2003/050085 discloses crystalline forms R, form VI, form Fa & form Je respectively.

U.S. Pat. No. 5,969,156 further discloses that the form-I possess more favorable filtration and drying characteristics than the known amorphous form of Atorvastatin calcium.

Atorvastatin calcium is a heat sensitive molecule and the reported crystalline forms, amorphous form requires prolonged periods for drying ranging from 18 hrs to 36 hrs to meet the ICH requirement for residual solvents.

Surprisingly, the present inventors have found a novel crystalline form of Atorvastatin calcium (2:1) herein designated as form M and also found a process for preparing the said crystalline form.

### SUMMARY OF THE INVENTION

Accordingly the present invention is directed to a novel crystalline Atorvastatin hemi-calcium form M its hydrates thereof and the processes for preparation.

Another object of the invention is to prepare crystalline Atorvastatin hemi-calcium form M from the known amorphous, crystalline forms or mixture of amorphous and crystalline forms.

Another object of the invention is to prepare crystalline Atorvastatin hemi-calcium form M which needs lesser time for drying to meet the ICH requirement for the residual solvents.

### BRIEF DESCRIPTION OF THE DRAWINGS

FIG. 1: XRD of crystalline Atorvastatin hemi-calcium Form-M

FIG. 2: DSC of crystalline Atorvastatin hemi-calcium Form-M

### DETAILED DESCRIPTION OF THE INVENTION

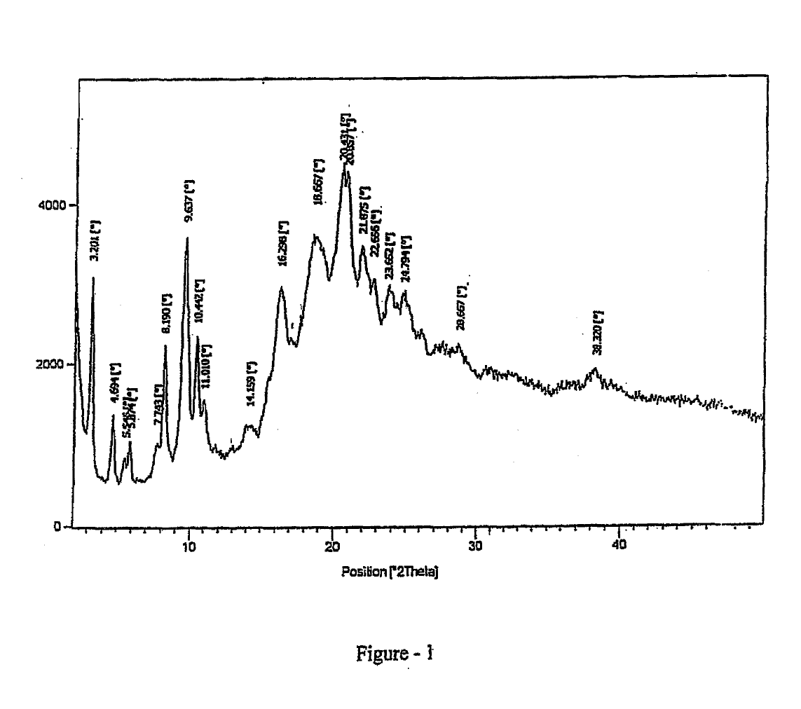

Thus in accordance with the present invention crystalline Atorvastatin hemi-calcium form M is characterized by the X-ray diffraction pattern as depicted in FIG. 1 having broad peaks at about 16.3 and 18.6 degrees 2θ and other peaks at 4.7, 5.5, 5.9, 8.2, 9.6, 10.4, 11.0, 20.3, 21.9 and 23.7±0.2 degrees 2θ.

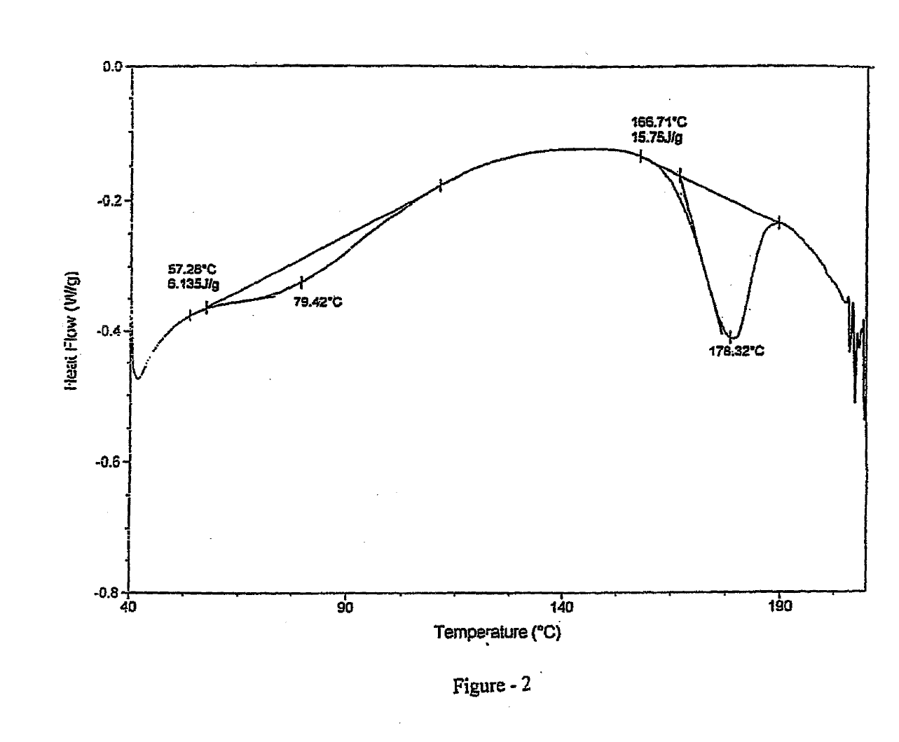

Crystalline Atorvastatin hemi-calcium Form M is further characterized by its DSC having 2 broad endotherms one at about 100° C. and the other at about 170-180° C. as depicted in FIG. 2. Crystalline Atorvastatin hemi-calcium form M exhibit water content about 1.0% to 6.0% w/w.

Crystalline Atorvastatin hemi-calcium form M can be prepared by treating Atorvastatin hemi-calcium amorphous form or form-I or mixture of amorphous and crystalline forms with methanol at room temperature to reflux temperature preferably at temperature of 15 to 35° C. for a period of 2 hrs to 30 hrs preferably for about 4 hrs to 18 hrs. The introduction of small quantity of form M as seeding will facilitates for quicker formation of crystalline Form M. After the precipitation of form M, it can be isolated as per the conventional methods. The wet material is dried at a temperature of about 30 to 65° C., preferably at 40 to 50° C. under vacuum for about 3 to 12 hrs.

The starting material Atorvastatin hemi-calcium amorphous form or form-I may be prepared by the prior art reported procedures.

Alternately, Atorvastatin hemi-calcium salt can be prepared by hydrolysis of (4R-Cis)-6-[2-(3-phenyl-4-(phenyl-carbomoyl)-(4-fluoro-phenyl)-5-(1-methylethyl)-pyrrol-1-yl)-ethyl]-2,2-dimethyl][1,3]dioxane-4-yl)-acetic acid tert.butyl ester with aqueous hydrochloric acid in methanol followed by treatment with aqueous sodium hydroxide. Reaction mass pH is adjustment to slightly basic or neutral with hydrochloric acid, concentration of reaction mass volume to about one third of its original volume by removal of solvent under vacuum followed by treatment with aqueous calcium acetate solution to get Atorvastatin hemi-calcium.

The prepared Atorvastatin hemi-calcium form M is characterized by its unique XRD, TGA and DSC.

The present invention is further illustrated with a few non-limiting examples.

### Example 1

### Preparation of Amorphous Atorvastatin Hemi-Calcium

(4R-CIS)-6-[2-(3-phenyl-4-(phenyl-carbomoyl)-(4-fluoro-phenyl)-5-(1-methylethyl)-pyrrol-1-yl)-ethyl]-2,2-dimethyl][1,3]dioxane-4-yl)-aceticacid tert.butyl ester (50 gm) is suspended in methanol (1000 ml), and maintained for 10 min. at temperature of 35° C. Reaction mass is cooled to 20-25° C. and 1N Hydrochloric acid (106 ml) is slowly added over 30 min. The reaction mass is maintained at 20-25° C. for 6 hrs, 10% sodium hydroxide solution (100 ml) is added and maintained for 3.5 hrs at the same temperature. Reaction mass pH is adjusted to 7.6 with 6N hydrochloric acid and treated with activated carbon (3.7 gm). Reaction mass is filtered and concentrated to a volume of about ⅓ of its original volume at temperature below 45° C. under vacuum. To the concentrated mass water (500 ml) and aqueous calcium acetate solution (6.5 gm in 50 ml water) are added at temperature of 25-30° C. over 30 min. Maintained the reaction mass at temperature of 25-30° C. for 4 hrs. Product is filtered; the wet cake is washed with 25% aq. Methanol (50 ml) and dried at temperature of 40-45° C. under vacuum for 6 hrs.

The dry weight of Atorvastatin hemi-calcium is 44 gm
Moisture content: 6.1% w/w,

### Example-2

### Preparation of Atorvastatin Hemi-Calcium Form M

Atorvastatin hemi-calcium (40 gm) is dissolved in methanol (160 ml) at temperature of 25-30° C. The obtained clear solution is treated with activated carbon (4 gm) at temperature of 25-30° C., for 30 min. Filtered the reaction mass and washed with methanol (40 ml). The clear filtrate is collected, cooled to 20-25° C., seeded with Atorvastatin hemi-calcium form M (0.4 gm) at temperature of 20-25° C. and maintained at temperature of 20-25° C. for 16 hrs. Reaction mass is diluted with methanol (160 ml) and maintained for further 4 hrs. The precipitated product is filtered; wet cake is washed with methanol (40 ml) and dried at 40-45° C. under vacuum for 4 hrs.

Dry weight of Atorvastatin hemi-calcium form M is 24 gm
Moisture content: 1.1% w/w and methanol content is 94 ppm.

### Example-3

### Preparation of Atorvastatin hemi-calcium form M

Atorvastatin hemi-calcium form M is prepared from Atorvastatin hemi-calcium form I by following the same procedure given in example-2.

1. Crystalline form M of Atorvastatin hemi-calcium.

2. Crystalline form M of Atorvastatin hemi-calcium which exhibits characteristic XRD pattern as depicted in FIG. 1.

3. Crystalline form M of Atorvastatin hemi-calcium which exhibits a characteristic XRD pattern with characteristic peaks at 16.3, 18.6, 4.7, 5.5, 5.9, 8.2, 9.6, 10.4, 11.0, 20.3, 21.9, 23.7±0.2 degrees 2θ.

4. Crystalline form M of Atorvastatin hemi-calcium which exhibits one broad endotherm at below 100° C. and a second endotherm at 170-180° C. in DSC.

5. A process for preparation Crystalline form M of Atorvastatin hemi-calcium comprising the steps of:
a) dissolving Atorvastatin hemi-calcium in methanol;
b) maintaining the solution for a period to crystallize form M; and
c) recovering the form M.

6. The process as claimed in claim 5, wherein the Atorvastatin hemi-calcium 25 used is step a) is in amorphous form.

7. The process as claimed in claim 5, wherein the Atorvastatin hemi-calcium used in step a) is crystalline form.

8. The process as claimed in claim 5, wherein the Atorvastatin hemi-calcium used in step a) is mixture of amorphous and crystalline forms.

9. The process as claimed in claim 5, wherein the solution of step b) is maintained for a period of about 2 to 30 hrs.

10. The process as claimed in claim 5, wherein the solution of step e is maintained for a period of about 4 to 18 hrs.

11. The process as claimed in claim 5, wherein the solution of step b) is maintained at a temperature of about 15° C. to 65° C.

12. The process as claimed in claim 5, wherein the solution of step b) is maintained at a temperature of about 15° C. to 35° C.

13. The process as claimed in claim 5, wherein the isolated form M is further dried at about 35 to 65° C.

14. The process as claimed in claim 5, wherein the isolated form M is further dried at about 35 to 50° C.

15. The process of claim 5, further comprising the step ^{cc} of seeding the dissolved Atorvastatin with Atorvastatin hemi-calcium form M.

16. The process as claimed in claim 13, wherein the drying is carried out for about 4 to 12 hrs.

17. The process as claimed in claim 14, wherein the drying is carried out for about 4 to 12 hrs.
